(19) United States
(12) Patent Application Publication
(10) Pub. No.: US 2012/0301452 A1
(43) Pub. Date: Nov. 29, 2012

(54) PROBIOTIC MICROORGANISMS AS ACTIVE AGENTS FOR ENHANCING THE RADIANCE OF THE SKIN'S COMPLEXION

(75) Inventors: Audrey Gueniche, Rueil Malmaison (FR); Isabelle Castiel, Nice (FR)

(73) Assignees: NESTEC S.A., Vevey (CH); L'OREAL, Paris (FR)

(21) Appl. No.: 13/514,872

(22) PCT Filed: Dec. 7, 2010
(86) PCT No.: PCT/IB2010/055629

§ 371 Date: Jul. 24, 2012

Related U.S. Application Data
(60) Provisional application No. 61/282,208, filed on Dec. 30, 2009.

(30) Foreign Application Priority Data
Dec. 8, 2009 (FR) 0958750

(51) Int. Cl. A61K 8/99; A61P 17/00; A61Q 11/00; A61K 35/74; A61K 35/66

(52) U.S. Cl. 424/93.45; 424/93.1; 424/780

(57) ABSTRACT

The invention relates to the use of at least one probiotic microorganism as an active agent for preventing and/or treating impaired radiance of the skin's complexion.

The present invention relates to the use of probiotic microorganisms for improving the radiance of the complexion, and especially for giving a luminous aspect to the complexion of the facial skin.

More particularly, the invention relates to the use of a probiotic microorganism, especially of the genus Lactobacillus sp., as an active agent that is useful for treating and/or preventing impaired radiance of the skin's complexion. The invention also relates to a process for making the skin's complexion more radiant, or even more luminous.

Radiance of the complexion is an indication of the healthy condition of the skin, especially of young skin.

Many external or internal factors, especially as indicated later, may generate imperfections on the skin and/or give rise to a blurred or non-uniform complexion. These complexion impairments, which are often of multifactor origin, are an increasingly frequent reason for consultation in beauty salons or dermatology surgeries.

The radiance of the skin's complexion may be affected by many external or internal factors. Among the extrinsic factors, mention may be made of exposure to sunlight, exposure to temperature and humidity variations, or exposure to pollutants. Among the intrinsic factors affecting the radiance of the skin's complexion, mention may be made of stress, tiredness, hormonal changes, dehydration of the epidermis or impairment of the skin's barrier function, or even ageing. These extrinsic and intrinsic factors have a tendency to blur the complexion, making it non-uniform, dull and waxy, or even sickly, and to promote or even worsen the presence of skin imperfections.

In their mildest form, these impairments affect almost everyone, and their frequency is maximal at the age of puberty. However, they may appear from the age of 7 to 9 and may continue beyond the age of 40, and may especially beyond the age of 60. Thus, it is common to have skin imperfections, especially on the face, and a blurry, dull and/or non-uniform complexion even after the age of 25.

In their most serious form, these impairments may occasionally have incapacitating psychosocial consequences, which may lead to isolation, or even depression or unemployment, in the case of individuals who are thereby affected.

There is thus a real need to be able to prevent, reduce or treat these skin complexion impairments.

WO 2006/104 730 describes a food supplement comprising probiotic microorganisms for improving the appearance of the skin, and preventing signs, especially age-related signs, on the skin, such as the appearance of a rough aspect or pallor.

However, it appears that none of the solutions proposed in the prior art proves to be satisfactory for preventing and/or treating impaired radiance of the skin's complexion.

Thus, there is a need for novel active agents that are useful for preventing and/or treating loss of radiance of the skin's complexion.

There is also a need for novel active agents that can give the skin a luminous complexion.

There is also a need for novel active agents for preventing and/or treating skin imperfections.

The object of the present invention is to satisfy these needs.

According to a first subject, the present invention relates to a cosmetic use of at least one probiotic microorganism, especially of the genus Lactobacillus sp., as an active agent that is useful for treating and/or preventing impaired radiance of the skin's complexion.

The inventors have observed, surprisingly, that the administration of a food supplement comprising a probiotic microorganism, especially of the genus Lactobacillus sp., in particular Lactobacillus paracasei and more particularly Lactobacillus paracasei ST11, can substantially reduce skin imperfections, prevent and/or treat a blurry and/or non-uniform and/or dull skin complexion, or give the skin a significantly more radiant and luminous complexion.

According to one embodiment, the impairments of radiance of the skin's complexion under consideration in the present invention are not age-related, and in particular are not signs of ageing of the skin.

The use of probiotic microorganisms according to the invention advantageously makes it possible to reinforce the skin's barrier function properties, thus promoting the integrity of the skin and maintaining its radiance.

According to another advantage, a use according to the invention makes it possible to optimize the assimilation of the nutrients supplied by food in the intestinal mucosae and to promote the supply of nutrients that are essential to cell metabolism. Synthesis of the various functional and structural components of the skin is thus promoted, allowing maintenance of the skin's homeostasis and reducing the loss of radiance of the complexion, or even reinforcing the skin's radiance and luminosity.

According to another preferred embodiment, a probiotic microorganism that is suitable for use in the invention as an active agent that is useful for treating and/or preventing impaired radiance of the skin's complexion may advantageously be a Lactobacillus sp., especially as defined hereinbelow.

According to one embodiment, a subject of the present invention is also a cosmetic process for preventing and/or treating impaired radiance of the skin's complexion, comprising the administration of at least one probiotic microorganism, especially of the genus Lactobacillus sp., especially as defined hereinbelow.

According to the invention, the term “skin” is intended to denote all of the epidermis of an individual, in particular of a human being, and more particularly the skin of the neckline, the neck and the face, especially facial skin.

According to one embodiment, a use according to the invention advantageously makes it possible to prevent and/or treat loss of radiance of the skin's complexion.

As indicated previously, the radiance of the skin's complexion may be adversely affected by the abovementioned intrinsic or extrinsic factors.

The skin's complexion reflects the healthy condition of the skin.

A luminous, radiant, or even glowing complexion is indicative of skin in good condition, whose properties and barrier functions are perfectly regulated.

The use of a probiotic microorganism in accordance with the invention thus advantageously makes it possible to regulate the skin's homeostasis so as to give the skin a luminous, more radiant or even more glowing complexion.

According to one embodiment, a use in accordance with the invention advantageously makes it possible to prevent and/or treat a blurry, dull and/or non-uniform skin complexion.

According to another embodiment, a use in accordance with the invention advantageously makes it possible to prevent and/or treat skin imperfections, chosen in particular from spots, dartres, dyschromia, age marks, blackheads and/or melasma.

According to another embodiment, a use in accordance with the invention advantageously makes it possible to prevent and/or treat skin imperfections chosen from spots, dartres, age marks, blackheads and/or melasma.

According to another embodiment, a use in accordance with the invention advantageously makes it possible to prevent and/or treat a waxy, yellowish or even sickly complexion.

According to another embodiment, the complexion impairments under consideration in the present invention are not related to greasy skin or skin prone to greasiness, or to acneic skin.

According to yet another embodiment, the skin under consideration in the invention is not aged skin or greasy skin or skin prone to greasiness.

According to one embodiment, a probiotic microorganism that is suitable for use in the invention may be administered orally, topically or parenterally, and in particular orally.

A use of a microorganism according to the invention necessarily takes place in an effective amount, i.e. an amount that allows the probiotic microorganism to display its active properties with regard to the impairments of radiance of the skin's complexion that are to be prevented and/or treated.

For the purposes of the present invention, the term “prevent” means at least partly reducing the risk of manifestation of a given phenomenon, i.e., in the present invention, impairment of the skin's complexion. A partial reduction implies that the risk remains, but to a lesser extent than before implementing the invention.

Microorganisms

For the purposes of the present invention, the term “probiotic microorganism” means a live microorganism, which, when consumed in adequate amount, has a positive effect on the health of its host (“Joint FAO/WHO Expert Consultation on Evaluation of Health and Nutritional Properties of Probiotic in Food Including Powder Milk with Live Lactic Acid Bacteria, 6 Oct. 2001”), and which may in particular improve the intestinal microbial balance.

According to one embodiment, a probiotic microorganism according to the invention may be used in an isolated or purified form, i.e. not mixed with one or more compound(s) liable to be associated with it in its medium of origin.

According to another embodiment, a probiotic microorganism according to the invention may be used in a live, semi-active, inactivated or dead form.

According to one advantageous embodiment of the invention, a probiotic microorganism may advantageously be used in inactivated or dead form.

A probiotic microorganism administered topically may advantageously be used in inactivated or dead form.

A probiotic microorganism administered orally may advantageously be used in live form.

For the purposes of the invention, an “inactivated” microorganism is a microorganism that is no longer capable, temporarily or definitively, of forming colonies in culture. For the purposes of the invention, a “dead” microorganism is a microorganism that is no longer capable, definitively, of forming colonies in culture. Dead or inactivated microorganisms may have intact or ruptured cell membranes. Thus, the term “inactivated” also denotes microorganism extracts and lysates as detailed hereinbelow. Dead or inactivated microorganisms may be produced via any method known to those skilled in the art.

According to one advantageous embodiment, the probiotic microorganisms used according to the invention are at least partly inactivated or dead.

The expression “probiotic microorganisms that are at least partly inactivated or dead” denotes a preparation of probiotic microorganisms in accordance with the invention comprising at least 80%, in particular at least 85%, more particularly at least 90%, or even at least 95%, or at least 99% of inactivated or dead probiotic microorganisms relative to the total amount of non-inactivated live probiotic microorganisms contained in the initial preparation before being subjected to a process to inactivate or kill the microorganisms.

The degree of inactivation or of death obtained depends on the application conditions of the method used, which are adjusted by a person skilled in the art according to the degree of inactivation or death to be obtained.

According to one embodiment, the invention comprises the use of a preparation comprising 100% of inactivated or dead probiotic microorganisms.

An inactivated probiotic microorganism that is suitable for use in the invention may be prepared by irradiation, heat inactivation or lyophilization of a microorganism preparation. These methods are known to those skilled in the art.

More particularly, the inactivation of probiotic microorganisms by irradiation may comprise the use of gamma rays, x-rays, exposure to UV or heat, or a pressure reduction. The type of treatment, the intensity, the dose and the exposure time are adjusted by a person skilled in the art according to the amount and nature of the probiotic microorganisms to be inactivated.

According to one preferred embodiment variant, a probiotic microorganism that is suitable for use in the invention is used in an inactivated form obtained by irradiation, especially gamma irradiation.

Inactivation by lyophilization may be performed via any method known in the field. Advantageously, probiotic microorganisms inactivated by lyophilization may be recultured.

Heat inactivation may be performed by incubating the probiotic microorganisms of the invention for a prolonged period of time, for example at least two hours, at 170° C. Heat inactivation may also be performed by autoclaving, by subjecting the probiotic microorganisms of the invention to a temperature of 121° C. for a period of at least 20 minutes and at an atmospheric pressure of 2 bar.

Alternatively, the heat inactivation may be performed by subjecting the probiotic microorganisms to a freezing temperature, for a prolonged period of time.

A probiotic microorganism according to the invention may be used in whole form, i.e. essentially in its native form, or in the form of extracts or lysates of disintegrated suspensions comprising fractions and/or metabolites of this microorganism.

For the purposes of the invention, the term “metabolite” denotes any substance derived from the metabolism of the microorganisms, and especially secreted by the microorganisms under consideration according to the invention and also endowed with efficacy for the treatment and/or prevention of loss of radiance of the skin's complexion.

For the purposes of the invention, the term “fraction” more particularly denotes a fragment of the said microorganism that is endowed with efficacy for the treatment and/or prevention of loss of radiance of the skin's complexion, by analogy with the said whole microorganism.

An extract or lysate that is suitable for use in the invention may be prepared from the probiotic microorganisms at the end of the growth phase.

According to one preferred embodiment, a probiotic microorganism that is suitable for use in the invention may be used in the form of a lysate.

For the purposes of the invention, a “lysate” commonly denotes a material obtained after the destruction or dissolution of biological cells via a phenomenon known as cell lysis, thus giving rise to the release of the intracellular biological constituents naturally contained in the cells of the microorganism under consideration.

For the purposes of the present invention, the term “lysate” is used without preference to denote the whole lysate obtained via lysis of the microorganism under consideration or only a fraction thereof.

The lysate used is thus totally or partially formed from the intracellular biological constituents and from the constituents of the cell walls and membranes.

Advantageously, a lysate used for the invention may be the whole lysate obtained via lysis of the microorganism under consideration.

This cell lysis may be accomplished via various techniques, such as an osmotic shock, a heat shock, via ultrasonication, or alternatively under a mechanical stress of centrifugation type.

More particularly, this cell lysate may be obtained according to the technique described in U.S. Pat. No. 4,464,362, and especially according to the following protocol.

In particular, a lysate of the invention may be obtained via ultrasonic disintegration of a medium comprising probiotic microorganisms in suspension in order to release therefrom the cytoplasmic fractions, the cell wall fragments and the products derived from metabolism. All the components in their natural distribution are then stabilized in a weakly acidic aqueous solution.

A microorganism, an extract or a lysate may be used in various forms, such as a solution, a culture supernatant, a powder, optionally lyophilized, or a concentrate.

According to one embodiment, a probiotic microorganism that is suitable for use in the invention may be chosen from Lactobacillus sp., Bifidobacterium sp., Cocci, yeasts and sporulating bacteria, and mixtures thereof.

According to one embodiment, a microorganism that is suitable for use in the invention is preferentially chosen from:
ascomycetes: especially Saccharomyces, especially S. cerevisiae or S. boulardii, Yarrowia, especially Y. Lipolytica, Kluyveromyces, Torulaspora, Schizosaccharomyces pombe, Debaromyces, Candida, Pichia, Aspergillus, especially K. lactis and Penicillium,
sporulating bacteria: especially Bacillus sp., especially B. cereus var toyo or B. subtilis, B. coagulans, B. licheniformis, Escherichia sp., especially E. coli strain nissle, Propionibacterium sp., especially P. freudenreichii, Bacteroides sp., Fusobacterium sp., and Weissella sp.,
Cocci: especially Melissococcus sp., Staphylococcus sp., especially S. carnosus or S. xylosus, Peptostrepococcus sp., Pediococcus sp., especially P. acidilactici, Micrococcus sp., Leuconostoc sp., especially L. mesenteroides subsp dextranicum, Aerococcus sp., Oenococcus sp., Enterococcus sp., especially E. faecalis or E. faecium, Lactococcus sp., especially L. lactis subsp lactis or L. cremoris, Sporolactobacillus sp., especially S. inulinus, and Streptococcus sp., especially S. salivarius subsp. thermophilus or S. thermophilus,
Lactobacillus species: especially L. paracasei, L. acidophilus, L. amylovorus, L. casei, L. rhamnosus, L. brevis, L. crispatus, L. delbrueckii subsp. lactis, L. bulgaricus, L. fermentum, L. helveticus, L. gallinarum, L. gasseri, L. johnsonii, L. plantarum, L. reuteri, L. salivarius, L. alimentarius, L. curvatus, L. casei subsp. casei, L. sake, and
bifidobacteria or Bifidobacterium species: B. adolescentis, B. animalis, B. bifidum, B. breve, B. lactis, B. longum, B. infantis, B. pseudocatenulatum,
and mixtures thereof.

More particularly it may be a probiotic microorganism chosen from Lactobacillus sp., Sporolactobacillus sp., Enterococcus sp., Lactococcus sp., Bacillus sp., Streptococcus sp., Pediococcus sp., Leuconostoc sp. or Bifidobacterium sp., and in particular chosen from Lactobacillus sp., Bifidobacterium sp., and mixtures thereof.

As examples of probiotic microorganisms that are suitable for use in the invention, mention may be made of Bifidobacterium adolescentis, B. animalis, B. bifidum, B. breve, B. lactis, B. longum, B. infantis, B. pseudocatenulatum, Lactobacillus acidophilus NCFB 1748, L. paracasei, L. amylovorus, L. casei (Shirota), L. rhamnosus strain GG, L. brevis, L. crispatus, L. bulgaricus, L. delbrueckii subsp. lactis, L. fermentum, L. helveticus, L. gallinarum, L. gasseri, L. johnsonii CNCM I-1225, L. plantarum, L. reuteri, L. salivarius, L. alimentarius, L. curvatus, L. casei subsp. casei, L. sake, Enterococcus faecalis, E. faecium, Lactococcus lactis, L. lactis subsp lactis, L. lactis subsp cremoris, Leuconostoc mesenteroides subsp. dextranicum, Pediococcus acidilactici, Sporolactobacillus inulinus, Streptococcus salvarius subsp. thermophilus, S. thermophilus, Staphylococccus carnosus, S. xylosus, Saccharomyces cerevisiae, S. boulardii, Bacillus cereus var toyo, B. cereus var subtilis, B. coagulans, B. licheniformis, Escherichia coli strain nissle, Propionibacterium freudenreichii, and mixtures thereof.

As more specific examples of probiotic microorganisms that are suitable for use in the invention, mention may be made advantageously of Bifidobacterium bifidum, B. infantis, B. longum, Lactobacillus acidophilus, L. alimentarius, L. casei subsp. casei, L. casei shirota, L. paracasei, L. curvatus, L. delbrueckii subsp. lactis, L. gasseri, L. johnsonii, L. reuteri, L. rhamnosus (Lactobacillus GG), L. sake, Lactococcus lactis, Streptococcus thermophilus, Staphylococccus carnosus, S. xylosus, and mixtures thereof.

More particularly as probiotic microorganisms that are suitable for use in the invention, mention may be made of Lactobacillus johnsonii or acidophilus, L. reuteri, L. rhamnosus, L. paracasei, L. casei, L. alimentarius, L. curvatus, L. delbrueckii subsp. lactis, L. gasseri, L. sake, Bifidobacterium bifidum, B. breve, B. longum, B. animalis, B. lactis, B. infantis, B. adolescentis, B. pseudocatenulatum, and mixtures thereof.

The species that are most particularly suitable for use in the invention are Lactobacillus johnsonii, L. paracasei, Bifidobacterium adolescentis, B. longum deposited, respectively, according to the Treaty of Budapest, at the Institut Pasteur (28 rue du Docteur Roux, F-75024 Paris cedex 15) on Jun. 30, 1992, Jan. 12, 1999, Apr. 15, 1999 and Apr. 15, 1999 under the following designations CNCM I-1225, CNCM I-2116, CNCM I-2168 and CNCM I-2170, and the genus Bifidobacterium lactis (Bb 12) (ATCC27536) or Bifidobacterium longum (BB536). The strain of Bifidobacterium lactis (ATCC27536) may be obtained from Hansen (Chr. Hansen A/S, 10-12 Boege Alle, P.O. Box 407, DK-2970 Hoersholm, Denmark).

Advantageously, a microorganism that is suitable for use in the invention may be a lactic acid probiotic microorganism, which produces lactic acid by fermentation of sugar.

According to one preferred embodiment, a probiotic microorganism that is suitable for use in the invention, for example topically or orally, and especially orally, may in particular be a microorganism of the genus Lactobacillus sp.

Preferably, a microorganism of the genus Lactobacillus sp. that is suitable for use in the invention may be chosen from Lactobacillus johnsonii, L. acidophilus, L. reuteri, L. paracasei and L. casei, and mixtures thereof.

According to one preferred embodiment, a microorganism that is suitable for use in the invention may be chosen from Lactobacillus paracasei, L. johnsonii and L. acidophilus and mixtures thereof.

According to one preferred embodiment, a microorganism that is suitable for use in the invention may be a Lactobacillus paracasei.

According to another preferred embodiment, a microorganism that is suitable for use in the invention may in particular be the strain Lactobacillus paracasei ST11 deposited according to the Treaty of Budapest at the Institut Pasteur (28 rue du Docteur Roux, F-75024 Paris cedex 15) on Jun. 30, 1992 under the designation CNCM I-2116.

According to one embodiment, a probiotic microorganism that is suitable for use in the invention is used orally, parenterally or topically.

The term “parenteral” route means a route other than the oral and topical routes. A parenteral route that is suitable for use in the invention may be, for example, the nasal route.

According to one preferred embodiment, a probiotic microorganism that is suitable for use in the invention is used topically.

The topical route advantageously makes it possible to obtain local action with regard to the desired effect.

According to another preferred embodiment, a probiotic microorganism that is suitable for use in the invention is used orally or parenterally, and in particular orally.

The oral route or the parenteral route advantageously makes it possible to obtain a global action with regard to the desired effect.

A microorganism of the invention may be formulated in a composition in a proportion of at least 0.0001% expressed as dry weight, in particular in a proportion from 0.0001% to 30%, in particular in a proportion from 0.001% to 20% and more particularly in a proportion from 0.01% to 15% by weight, in particular from 0.1% to 10% by weight and especially from 1% to 5% by weight relative to the total weight of the composition containing it.

In general, a composition according to the invention intended to be administered orally may comprise for the live microorganisms from 10³ to 10¹⁵ cfu/g, in particular from 10⁵ to 10¹⁵ cfu/g and more particularly from 10⁷ to 10¹² cfu/g of live microorganisms per gram of composition, or equivalent doses calculated for inactivated or dead microorganisms or for microorganism fractions or for produced metabolites.

In particular, in a composition administered orally, the corresponding microorganism and/or fraction and/or metabolite concentration may be adjusted so as to correspond to doses, expressed as microorganism equivalent, ranging from 5×10⁵ to 10¹³ cfu/day and in particular from 10⁸ to 10¹¹ cfu/day.

A composition for topical application according to the invention may generally comprise from 0.0001% to 30% by weight of microorganisms per gram of composition, in particular a proportion of from 0.001% to 20%, more particularly a proportion of from 0.01% to 15% by weight, in particular from 0.1% to 10% by weight and especially from 1% to 5% by weight of microorganisms relative to the total weight of the composition containing it.

In the particular case of topical administration, it may be advantageous to use microorganisms in inactivated or even dead form, especially in the form of an extract or a lysate.

A microorganism may also be included in a composition in the form of fractions of cell components or in the form of metabolites, in particular in the form of a lysate. The microorganism(s), metabolite(s) or fraction(s) may also be introduced in the form of a lyophilized powder, a culture supernatant and/or, where appropriate, in a concentrated form.

When a composition comprises metabolites, the contents of metabolites in the compositions correspond substantially to the contents that may be produced by 10³ to 10¹⁵ cfu, in particular 10⁵ to 10¹⁵ cfu and more particularly 10⁷ to 10¹² cfu of live microorganisms per gram of composition.

Expression of the amount of metabolites or fractions of a microorganism in “cfu”, or of dead microorganisms, is intended to denote the amount of this microorganism used for the preparation of the metabolites or fractions of this microorganism.

Compositions

A probiotic microorganism that is suitable for use in the invention is advantageously formulated in a composition that may be in any galenical form normally available for the selected mode of administration.

A composition according to the invention comprises a physiologically or pharmaceutically acceptable medium.

A composition according to the invention may be administered orally, parenterally, especially subcutaneously or intradermally, or topically.

Preferably, a composition of the invention may be administered topically.

According to one embodiment, a topical composition according to the invention may advantageously be formulated in any galenical form that is suitable for caring for the skin and mucous membranes and may be in the form of ointments, creams, milks, pomades, powders, impregnated pads, solutions, gels, sprays, lotions or suspensions. They may also be in the form of microspheres or nanospheres or lipid or polymer vesicles or polymer patches and hydrogels allowing controlled release. These topical compositions may be either in anhydrous form or in aqueous form according to the dermocosmetic indication.

A composition intended for topical administration may be an aqueous, aqueous-alcoholic or oily solution, a solution or dispersion of the lotion or serum type, an emulsion of liquid or semi-liquid consistency of the milk type, obtained by dispersing a fatty phase in an aqueous phase (O/W) or conversely (W/O), a suspension or an emulsion of soft, semi-solid or solid consistency, of the cream type or of the aqueous or anhydrous gel type, a multiple emulsion (W/O/W or O/W/O), a microemulsion, a nanoemulsion, a preparation of microcapsules, a preparation of microparticles, a vesicular dispersion of ionic and/or nonionic type, or a wax/aqueous-phase dispersion.

According to one preferred embodiment, a topical composition may be in the form of a solution, a cream, a gel, an emulsion, a mousse or an aerosol composition containing a propellant.

According to one preferred embodiment, a topical composition may also be in the form of a transdermal system for active or passive release of the active agent(s) transdermally, for example of patch or gel patch (hydrogel) type.

These compositions are prepared according to the usual methods.

A composition according to the invention may constitute a composition for treating or caring for the skin or the scalp, or an antisun or artificial-tanning composition, or alternatively a skin-cleansing or makeup-removing product, a deodorant product or a fragrancing compound.

Such a composition may then be uncoloured or weakly coloured, and may optionally contain additional cosmetic or dermatological active agents, especially as indicated hereinbelow. It may then be used as a care base for the skin or the lips, for example in the form of a lip balm, protecting the lips against the cold and/or sunlight and/or the wind, or as a day or night care cream for facial and/or bodily skin.

It may also be in the form of a medicated or unmedicated, colouring or non-colouring shampoo, or a hair conditioner.

A composition according to the invention may also constitute a coloured cosmetic composition and especially a makeup composition for the skin and/or mucous membranes. In particular, such a composition may be a foundation, a blusher, a face powder, an eyeshadow, a concealer compound in stick form, a lipstick or a lip gloss, optionally with care or treating properties. Preferably, it may be a coloured (beige or green) makeup composition for correcting the colour of the complexion.

A composition administered topically may especially constitute a cleansing, protective, treating or care cream, a skincare lotion, gel or mousse, a cleansing or disinfecting lotion, a bath composition or a deodorant composition.

A composition according to the invention may also consist of a solid preparation constituting a cleansing soap or bar.

When a composition of the invention is an emulsion, the proportion of the fatty phase may range from 5% to 80% by weight and preferably from 10% to 50% by weight relative to the total weight of the composition. The oils, emulsifiers and co-emulsifiers used in the composition in emulsion form are chosen from those conventionally used in cosmetics and/or dermatology. The emulsifier and the co-emulsifier may be present in the composition in a proportion ranging from 0.3% to 30% by weight and preferably from 0.5% to 20% by weight relative to the total weight of the composition.

When the composition of the invention is an oily gel or solution, the fatty phase may represent more than 90% of the total weight of the composition.

In a known manner, galenical forms intended for topical administration may also contain adjuvants that are common in the cosmetic, pharmaceutical and/or dermatological field, such as hydrophilic or lipophilic gelling agents, hydrophilic or lipophilic active agents, preserving agents, antioxidants, solvents, fragrances, fillers, screening agents, odour absorbers and dyestuffs. The amounts of these various adjuvants are those conventionally used in the field under consideration, for example from 0.01% to 20% of the total weight of the composition. Depending on their nature, these adjuvants may be introduced into a fatty phase and/or into the aqueous phase.

As fatty substances that may be used in the invention, mention may be made of mineral oils, for instance hydrogenated polyisobutene and liquid petroleum jelly, plant oils, for instance the liquid fraction of shea butter, sunflower oil and apricot kernel oil, animal oils, for instance perhydrosqualene, synthetic oils, especially purcellin oil, isopropyl myristate and ethylhexyl palmitate, unsaturated fatty acids, and fluoro oils, for instance perfluoropolyethers. Fatty alcohols, fatty acids, for instance stearic acid, and, for example, waxes, especially paraffin wax, carnauba wax and beeswax, may also be used. Silicone compounds may also be used, for instance silicone oils, for example cyclomethicone and dimethicone, and silicone waxes, resins and gums.

As emulsifiers that may be used in the invention, examples that may be mentioned include glyceryl stearate, polysorbate 60, the mixture of cetylstearyl alcohol/cetylstearyl alcohol oxyethylenated with 33 mol of ethylene oxide, sold under the name Sinnowax AO® by the company Henkel, the mixture of PEG-6/PEG-32/glycol stearate sold under the name Tefose® 63 by the company Gattefossé, PPG-3 myristyl ether, silicone emulsifiers such as cetyldimethicone copolyol, and sorbitan monostearate or tristearate, PEG-40 stearate and oxyethylenated (20 OE) sorbitan monostearate.

As solvents that may be used in the invention, mention may be made of lower alcohols, especially ethanol and isopropanol, and propylene glycol.

The composition of the invention may also advantageously contain a spring and/or mineral water, chosen especially from Vittel water, waters from the Vichy basin, and Roche Posay water.

Hydrophilic gelling agents that may be mentioned include carboxylic polymers such as carbomer, acrylic copolymers such as acrylate/alkylacrylate copolymers, polyacrylamides and especially the mixture of polyacrylamide, C₁₃₋₁₄ isoparaffin and Laureth-7 sold under the name Sepigel 305® by the company SEPPIC, polysaccharides, for instance cellulose-based derivatives such as hydroxyalkylcelluloses and in particular hydroxypropylcellulose and hydroxyethylcellulose, natural gums such as guar gum, locust bean gum and xanthan gum, and clays.

Lipophilic gelling agents that may be mentioned include modified clays, for instance bentones, metal salts of fatty acids, for instance aluminium stearates, and hydrophobic silica, or alternatively ethylcellulose and polyethylene.

In the case of a composition in accordance with the invention for oral administration, the use of an ingestible support is preferred.

The ingestible support may be of diverse nature according to the type of composition under consideration.

Tablets, gel capsules or lozenges, suspensions, oral supplements in dry form and oral supplements in liquid form are thus especially suitable for use as food supports.

Milk, yoghurt, cheese, fermented milks, milk-based fermented products, ice creams, cereal-based products or fermented cereal-based products, milk-based powders, baby and infant formulas, food products of confectionery type, chocolate, cereals or animal feed, in particular for pets, are also suitable for use as food supports.

The term “oral composition” means, for example, nutritional, nutraceutical, cosmeceutical or pharmaceutical compositions, comprising at least one probiotic microorganism according to the invention.

Formulation of the oral compositions according to the invention may be performed via any usual process known to those skilled in the art for producing drinkable solutions, sugar-coated tablets, gel capsules, gels, emulsions, tablets to be swallowed or chewed, wafer capsules, especially soft or hard wafer capsules, granules to be dissolved, syrups, solid or liquid foods, and hydrogels allowing controlled release.

In particular, a probiotic microorganism according to the invention may be incorporated into any form of food supplement or enriched food, for example food bars, or compacted or loose powders. The powders may be diluted with water, with soda, with dairy products or soybean derivatives, or may be incorporated into food bars.

According to one embodiment, a composition according to the invention administered orally may be formulated in the form of sugar-coated tablets, gel capsules, gels, emulsions, tablets, wafer capsules, hydrogels, food bars, compacted or loose powders, liquid suspensions or solutions, confectioneries, fermented milks, fermented cheeses, chewing gum, toothpaste or spray solutions.

The oral compositions may be either in anhydrous form or in aqueous form according to the dermocosmetic indication.

A probiotic microorganism of the invention may moreover be formulated with the excipients and components that are common for such oral compositions or food supplements, i.e. especially fatty and/or aqueous components, humectants, thickeners, preserving agents, texturizers, flavour enhancers and/or coating agents, antioxidants and preserving agents.

Formulating agents and excipients for oral compositions, and especially for food supplements, are known in this field and will not be the subject of a detailed description herein.

According to the preferred indication of the use of a probiotic microorganism of the invention, a composition of the invention will be a cosmetic, dermatological or pharmaceutical composition.

In particular, the composition according to the invention may be a food composition for human consumption. It may in particular be a case of nutritional whole foods, drinks, mineral waters, soups, dietary supplements and replacement foods, nutritional bars, confectioneries, fermented or unfermented milk-based products, yoghurt products, milk-based powders, enteral nutritional products, baby and/or infant compositions, fermented or unfermented cereal-based products, ice creams, chocolate, coffee, or “culinary” products such as mayonnaise, tomato puree or salad dressings.

The composition according to the invention may also be intended for animals, especially pets, such as cats and dogs, and may be formulated in the form of feed or food supplements for animals.

According to one preferred embodiment, a probiotic microorganism that is suitable for use in the invention may be used parenterally, in particular subcutaneously or intradermally.

In such a use, a probiotic microorganism that is suitable for use in the invention may be conditioned in the form of an aqueous or non-aqueous sterile isotonic solution, in the form of a dispersion, a suspension or an emulsion prepared, where appropriate, just before administration, using a sterile powder, for example a lyophilized powder, which is then reconstituted in the form of an injectable sterile solution or a dispersion at the time of use.

The term “sterile” qualifies a formulation that is capable of ensuring the harmlessness required for intraepidermal and/or intradermal and/or subcutaneous administration.

A composition that is suitable for use in the invention may comprise any excipient usually used in the field of injectable sterile solutions.

The parenteral administration of a composition of the invention may be performed via any injection technique that is suitable for intraepidermal and/or intradermal and/or subcutaneous injection. Thus, such an administration may be performed by means of a needle usually used for performing an intraepidermal and/or intradermal and/or subcutaneous injection, suitable for mesotherapy.

Additional Active Agent

A probiotic microorganism according to the invention may advantageously be used in combination with an additional active agent, especially a cosmetic or pharmaceutical active agent.

Advantageously, such an additional cosmetic or pharmaceutical active agent may be intended for exerting a cosmetic, care or hygienic effect on the skin, the hair, the eyelashes, bodily hair and/or the scalp, and preferentially on the skin.

The additional active agents are chosen by a person skilled in the art such that they do not harm the effect of the probiotic microorganisms of the invention.

In particular, an additional active agent that is suitable for use in the invention may be chosen from active agents for reinforcing the cutaneous barrier.

According to another embodiment, active agents for preventing and/or treating skin complaints may be combined with a microorganism according to the invention.

As additional active agents that may be used, mention may be made of:
vitamins, such as vitamins A, B5, B6, B8, C, D, E or PP (vitamin B3 or niacin),
antioxidants, such as curcuminoids; carotenoids, especially a carotenoid chosen from β-carotene, lycopene, astaxanthin, zeaxanthin and lutein; polyphenol compounds, flavonoids such as catechins; proanthocyanidins, anthocyanins and PCOs (procyannidol oligomers); ubiquinones; coffee extracts containing polyphenols and/or diterpenes; chicory extracts; Ginkgo biloba extracts; proanthocyanidin-rich grape extracts; pimento extracts; soybean extracts,
minerals, such as zinc, calcium, magnesium, copper, iron, iodine, manganese, selenium or chromium (III),
sugars,
amino acids, especially sulfur-containing amino acids, such as glutathione precursors, taurine, and selenium-containing amino acids,
3 and 6 polyunsaturated fatty acids,
prebiotics, chosen especially from oligosaccharides, produced from glucose, galactose, xylose, maltose, sucrose, lactose, starch, xylan, hemicellulose, inulin, gums of acacia type, for example, or a mixture thereof. More particularly, the oligosaccharide comprises at least one fructo-oligosaccharide. More particularly, this prebiotic may comprise a mixture of fructo-oligosaccharide and inulin,
phytosterols, for instance resveratrol,
hesperidin, and
mixtures thereof.

According to one embodiment, proteins or protein hydrolysates, amino acids, polyols, especially C₂ to C₁₀ polyols, for instance glycerol, sorbitol, butylene glycol and polyethylene glycol, urea, allantoin, sugars and sugar derivatives, water-soluble vitamins, starch, and bacterial or plant extracts such as those of Aloe vera, may be used more particularly in the topical galenical forms as additional hydrophilic active agents.

According to another embodiment, retinol (vitamin A) and derivatives thereof, tocopherol (vitamin E) and derivatives thereof, ceramides, essential oils and unsaponifiable materials (tocotrienol, sesamine, gamma-oryzanol, phytosterols, squalenes, waxes and terpenes) may be used more particularly in the topical galenical forms as additional lipophilic active agents.

According to another embodiment, a topical galenical form that is suitable for use in the invention may comprise as additional active agent at least one anti-ageing active agent.

Such an anti-ageing active agent may be chosen especially from moisturizers, desquamating agents, agents for improving the barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting maturation of the horny envelope, NO-synthase inhibitors, peripheral benzodiazepine receptor (PBR) antagonists, agents for stimulating the energy metabolism of cells, tensioning agents, and agents for promoting the cutaneous capillary circulation.

According to yet another embodiment, a depigmenting agent or skin-bleaching agent may advantageously be used in the topical galenical forms as additional active agent.

Such a depigmenting agent or bleaching agent may be chosen especially from an extract of at least one mint, and more particularly from the genus Mentha piperita; vitamin C and derivatives thereof, for instance vitamins CG or CP and 3-O-ethyl vitamin C; polyphenols such as ellagic acid; ferulic acid; lucinol and derivatives thereof; kojic acid; α- and β-arbutin and derivatives thereof; hydroquinone; aminophenol derivatives; iminophenol derivatives; L-2-oxothiazolidine-4-carboxylic acid or procysteine, and salts and esters thereof; resorcinol and derivatives thereof; tranexamic acid and derivatives thereof; gentisic acid, homogentisate and methyl gentisate; dioic acid; calcium D-pantheteine sulfonate; lipoic acid; vitamin B3; linoleic acid and derivatives thereof; ceramides and homologues thereof; and plant extracts, in particular from liquorice, from mulberry, from skullcap, from Bacopa monnieri, from camomile, from bearberry, from the Aloe family (vera, ferox or bardensis), a kiwi fruit (Actinidia chinensis) juice, or an extract of Paeonia suffruticosa root, without this list being limiting.

According to yet another embodiment, a topical galenical form that is suitable for use in the invention may comprise as additional active agent at least one desquamating agent.

Such a desquamating agent may be chosen especially from α- and β-hydroxy acids, glycolic acid, citric acid, lactic acid, tartaric acid, malic acid, mandelic acid, salicylic acid and derivatives thereof, in particular 5-n-octanoylsalicylic acid; pyruvic acid, gluconic acid, glucuronic acid, oxalic acid, malonic acid, succinic acid, acetic acid, gentisic acid, cinnamic acid or azelaic acid; phenol, resorcinol; urea and derivatives thereof; oligofucoses; jasmonic acid and derivatives thereof; trichloroacetic acid; retinoids such as retinol or retinoic acid; adapalene; extract of Saphora japonica; resveratrol; enzymes involved in the desquamation or degradation of corneodesmosomes, such as glycosidases, stratum corneum chymotryptic enzyme (SCCE) or other proteases; mineral salt chelating agents such as EDTA; N-acyl-N,N′,N′-ethylenediaminetriacetic acid; aminosulfonic compounds and in particular (N-2-hydroxyethylpiperazine-N-2-ethane)sulfonic acid (HEPES); 2-oxothiazolidine-4-carboxylic acid (procysteine) derivatives; derivatives of α-amino acids such as glycine; honey; sugar derivatives such as O-octanoyl-6-D-maltose, O-linoleyl-6-D-glucose and N-acetylglucosamine; extracts of laminaria such as Laminaria saccharina and Laminaria ochroleuca, glyceryl trilactate, siliceous salicylate derivatives; and an extract of the flowers of ficus Opuntia indica, for instance Exfolactive® from Silab.

As additional active agents in an oral galenical form, any commonly used and/or permitted ingredient may also be considered.

By way of illustration, mention may be made of vitamins, minerals, essential fats, trace elements, polyphenols, flavonoids, phytoestrogens, antioxidants such as lipoic acid and coenzyme Q10, carotenoids, prebiotics, proteins and amino acids, monosaccharides and polysaccharides, amino-sugars, phytosterols and triterpene alcohols of plant origin.

This in particular concerns vitamins A, C, D, E and PP and B group vitamins, especially B5, B6 and B8.

Among the carotenoids, β-carotene, lycopene, lutein, zeaxanthin and astaxanthin are preferably chosen.

The minerals and trace elements particularly used are zinc, calcium, magnesium, copper, iron, iodine, manganese, selenium and chromium (III).

Among the polyphenols, polyphenols from grape, tea, olive, cocoa, coffee, apple, blueberry, elderberry, strawberry, cranberry and onion may be selected in particular.

Preferably, among the phytoestrogens, isoflavones in free or glycosylated form are selected, such as genistein, daidzein, glycitein or lignans, in particular those from flax and from Schizandra chinensis.

Preferably, among the amino acids that are suitable for use in the invention, taurine, threonine, cysteine, tryptophan or methionine, or peptides and proteins containing them, may be chosen.

Preferably, among the lipids that are suitable for use in the invention, lipids belonging to the group of oils containing monounsaturated and polyunsaturated fatty acids, such as oleic acid, linoleic acid, α-linolenic acid, γ-linolenic acid, stearidonic acid, long-chain fish omega-3 fatty acids such as EPA and DHA, conjugated fatty acids obtained from plants or animals such as CLA (Conjugated Linoleic Acid), may be chosen.

Thus, a composition intended for oral administration may also comprise at least one nutritional active agent chosen from lycopene, vitamin C, vitamin E and polyphenol compounds.

An oral composition of the invention may also comprise other nutritional active agents chosen from:
anti-ageing nutritional active agents, such as food antioxidants, nutrients with radical-scavenging properties and cofactors of antioxidant endogenous enzymes, vitamins A, C and E, carotenoids, xanthophylls, isoflavones, certain minerals such as zinc, copper, magnesium or selenium, lipoic acid, coenzyme Q10, superoxide dismutase (SOD) or taurine. Among the anti-ageing active agents, mention may be made especially of unsaponifiable fractions extracted from fats of plant origin, Aloe vera, native or hydrolysed marine collagen, and plant or marine oils rich in omega-3 and omega-6 fatty acids (including γ-linolenic acid),
photoprotective nutritional active agents such as antioxidants and free-radical scavengers: vitamins A, C and E, carotenoids, xanthophylls, certain minerals such as zinc, copper, magnesium or selenium, coenzyme Q10 and superoxide dismutase (SOD),
nutritional ingredients with moisturizing or immunomodulatory properties such as the extract of Polypodium leucotomos, plant or marine oils rich in omega-3 or omega-6 fatty acids, including gamma-linolenic acid.

According to another embodiment, a probiotic microorganism that is suitable for use in the invention may advantageously be used orally in combination with a depurative agent chosen especially, for example, from extracts of birch or of black radish.

According to another preferred embodiment, a probiotic microorganism that is suitable for use in the invention may be used parenterally, in particular subcutaneously or intradermally, in combination with at least one filling agent or an agent for stimulating fibroblast or keratinocyte proliferation, or an agent for stimulating the synthesis of dermal macromolecules.

Advantageously, a probiotic microorganism that is suitable for use in the invention may be used parenterally in combination with an agent chosen from extracts of Centella asiatica; asiaticosides and derivatives; synthetic peptides; peptides extracted from plants; plant hormones such as gibberellins and cytokinins; an extract of Saccharomyces cerevisiae; an extract of the alga Macrocystis pyrifera; retinoids and derivatives; oligopeptides and lipopeptides; lipoamino acids; a malt extract; extracts of blueberry or rosemary; lycopene; isoflavones, derivatives thereof or plant extracts containing them, extracts of red clover, of linseed, of kakkon or of sage; a peptide extract of seeds of leguminous plants (Pisum sativum); heparinoids; pseudodipeptides; an extract of lupin; an extract of buds from beech Fagus sylvatica; an extract of Salina zooplankton; phloroglucinol; extracts of walnut oil cakes; extracts of Solanum tuberosum; hyaluronic acid, especially with a weight-average molecular weight ranging from 50 000 to 3 000 000 daltons.

According to one embodiment, the invention relates to a cosmetic process for preventing and/or treating impaired radiance of the skin's complexion in the case of an individual in need thereof, comprising at least one step of administering to the said individual at least one probiotic microorganism, especially of the genus Lactobacillus sp.

A process according to the invention may comprise a step that consists in observing a reduction in or even the disappearance of the impaired radiance of the complexion, or alternatively an improvement in the radiance of the complexion.

A process according to the invention may especially be performed topically by administration of a topical cosmetic and/or dermatological composition as defined above.

Advantageously, a process of the invention comprising topical application may comprise the application of a composition comprising at least one probiotic microorganism in accordance with the invention, for example in the form of a mask on the skin.

Such an administration may be performed according to the usual techniques for using these compositions. For example, it may consist in applying creams, gels, sera or lotions to the skin or mucous membranes.

A topical cosmetic process according to the invention may, for example, be performed daily, for example at a rate of one administration per day or an administration twice a day, for example once in the morning and once in the evening.

A topical cosmetic process according to the invention may be performed over a period of time ranging from one week to several weeks, or even several months, this period moreover possibly being repeated after periods without treatment, for several months or even several years.

By way of example, the topical administration of a probiotic microorganism according to the invention may be repeated, for example, 2 to 3 times a day, or more, and generally over a prolonged period of at least 4 weeks, or even 4 to 15 weeks, with, where appropriate, one or more periods of stoppage.

A cosmetic process according to the invention may be performed orally, especially by administration of a food composition as defined above.

An effective amount of microorganism may be administered in a single dose per day or in fractional doses over the day, for example two to three times a day.

An oral cosmetic process may be performed over a time period ranging from one week to several weeks, or even several months, this period moreover possibly being repeated after periods without treatment, for several months or even several years.

By way of example, the oral administration of a probiotic microorganism according to the invention may be performed at a rate, for example, of 3 times a day or more, generally over a prolonged period of at least 4 weeks, or even 4 to 15 weeks, optionally comprising one or more periods of stoppage or being repeated after a period of stoppage.

### FIGURES

FIG. 1: illustrates the change in facial skin complexion over time, at D1, D29 and D57. The top graph represents the ST11 group and the bottom graph represents the placebo group. The results show a marked improvement in the complexion of the skin in the case of the individuals of the ST11 group (dark grey and intermediate grey bars, degrees 0 and 1 on a scale of 4). The results are expressed as a mean±confidence interval.

FIG. 2: illustrates the change in facial skin imperfections over time, at D1, D29 and D57. The top graph represents the ST11 group and the bottom graph represents the placebo group. The results show a very marked reduction in facial skin imperfections for the individuals of the ST11 group (dark grey and intermediate grey bars, degrees 0 and 1 on a scale of 3). The results are expressed as a mean±confidence interval.

In the description and in the examples that follow, unless otherwise mentioned, the percentages are weight percentages and the ranges of values worded in the form “between . . . and . . . ” include the stated lower and upper limits.

The ingredients are mixed together, before being formed, in the order and under conditions that may be readily determined by a person skilled in the art.

The content and nature of the ingredients used in the compositions of the invention are adjusted by a person skilled in the art so as not to substantially affect the properties required for the compositions of the invention.

The examples below are presented as non-limiting illustrations of the invention.

### EXAMPLES

### Example 1

### Effect of a Food Supplement Comprising Lactobacillus paracasei ST11 on the Radiance of the Skin's Complexion

Materials and Methods

The effect of a food supplement comprising Lactobacillus paracasei ST11 on the radiance of the skin's complexion was determined by performing a double-blind randomized controlled comparative single-centre study, on two parallel groups of 33 individuals: food supplement (ST11 group) vs placebo (placebo group).

The complexion radiance measurements were taken by clinical assessment by an evaluating dermatologist.

The individuals selected for the study are healthy males over 60 years old, having at the start of the study (D0) a score of 4 or 5 on the Densiscore® and, in principle, being low consumers of fermented products (less than 125 g/day).

During the study, the volunteers agreed not to consume any fermented products containing live bacteria (yoghurts, cottage cheeses, fermented milks, unpasteurized cheeses, etc.).

The food supplement was formulated in the form of a powder comprising live Lactobacillus paracasei NCC 2461 (ST11) at a total dose of 10⁹ cfu and maltodextrin. The powder is to be dispersed or dissolved in a drinkable liquid, preferably water.

The placebo was formulated in the form of a powder comprising only malto-dextrin. The powder is to be dispersed or dissolved in a drinkable liquid, preferably water.

The treatment was administered once a day, for 61 days. The measurements were taken on D-42 (preinclusion), D0, D1, D2, D3, D4, D5, D29, D30, D31, D32, D33, D57, D58, D59, D60 and D61.

The clinical score of the complexion of the face was determined visually at each visit by an investigating dermatologist, on a scale from 0 to 4 (0=very radiant; 1=radiant; 2=neither dull/nor radiant; 3=dull; 4=very dull).

The clinical score of the facial imperfections (spots, dartres, dyschromia, age marks and blackheads) was determined visually at each visit by an investigating dermatologist, on a scale from 0 to 4 (0=absence: no imperfections; 1=slight: a few imperfections (from 1 to 5); 2=moderate: from 5 to 12 imperfections; 3=severe: more than 12 and less than 25 imperfections; 4=extreme: very large number of imperfections).

Results

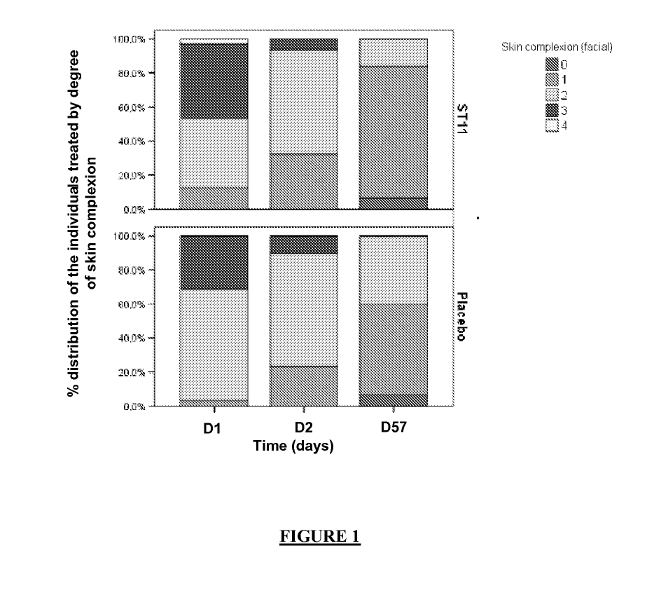

Facial Skin Complexion (FIG. 1)

The facial skin complexion for the two groups (ST11 and placebo) was comparable at D1 (p=0.4412).

On the other hand, the facial skin complexion becomes significantly more luminous and more radiant over time for the ST11 group than for the placebo group. The change in radiance of the complexion between D1 and D57 for the two groups is statistically significant in favour of the ST11 group (p=0.0408).

At the end of the supplementation, a 54% increase in luminosity of the complexion is observed for the individuals treated with a food supplement according to the invention (ST11 group) and of only 22% for the placebo group.

TABLE 1
Tests of intra-group and inter-group comparison (p-values) of the
change in facial skin complexion relative to D1 at all the times.

| | PLACEBO/ST11 |
|---|---|
| Time | (p value) |
| D1-D29 | 0.1624 |
| D1-D57 | 0.0408 |

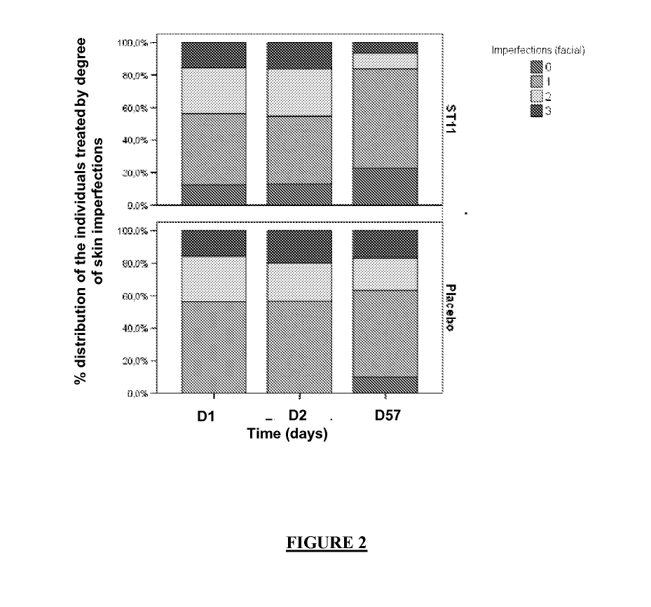

Facial Skin Imperfections (FIG. 2)

The skin imperfections evaluated were specially spots, dartres, dyschromia, age marks and blackheads.

The two groups, ST11 and placebo, appear comparable at D1 (p=0.5901).

On the other hand, the facial skin imperfections decrease significantly between D1 and D57 solely for the ST11 group. The change in facial skin imperfections between D1 and D57 is statistically significant in favour of the ST11 group (p=0.0175).

At the end of the supplementation, a 22% decrease in facial skin imperfections is observed for the individuals treated with a food supplement according to the invention (ST11 group), and of only 10% for the placebo group.

TABLE 2
Tests of intra-group and inter-group comparison (p-values) of the
change in facial imperfections relative to D1 at all the times.

| | PLACEBO/ST11 |
|---|---|
| Time | (p value) |
| D1-D29 | 0.8845 |
| D1-D57 | 0.0175 |

### CONCLUSION

The results obtained in the clinical study detailed above show that the oral administration of a food supplement comprising Lactobacillus paracasei (ST11) at a dose of 10⁹ cfu/day for two months makes it possible to obtain, on clinical assessment:
a significantly more radiant complexion (overall change in complexion between D1 and D57 for the ST11 group vs the placebo group, p=0.0408); and

a decrease in facial imperfections over time solely for the ST11 group (p<0.0001). The difference is statistically significant in favour of ST11 if the change is studied between D1 and D57 (p=0.0175) for the two treatments.

### Example 2

### Topical Compositions According to the Invention

### Example 2A

### Facial Lotion

| | weight % |
|---|---|
| Lyophilized powder of Lactobacillus paracasei ST11 | 5.00 |
| Lyophilized powder of Lactobacillus johnsonii | 5.00 |
| Anti-inflammatory agent | 0.05 |
| Antioxidant | 0.05 |
| Isopropanol | 40.0 |
| Preserving agent | 0.30 |
| Water | qs 100 |

### Example 2B

### Facial Care Gel

| | weight % |
|---|---|
| Powder of Lactobacillus paracasei ST11 | 5.00 |
| Hydroxypropylcellulose (Klucel H ® sold | 5.00 |
| by the company Hercules) | |
| Vitamin E | 1.00 |
| Antioxidant | 0.05 |
| Isopropanol | 40.00 |
| Preserving agent | 0.30 |
| Water | qs 100 |

### Example 2C

### Facial Care Milk

| | weight % |
|---|---|
| Lyophilized powder of Lactobacillus paracasei ST11 | 5.00 |
| Glyceryl stearate | 1.00 |
| Cetylstearyl alcohol/cetylstearyl alcohol oxyethylenated | 3.00 |
| with 30 mol of OE (Sinnowax AO ® sold by the company | |
| Henkel) | |
| Cetyl alcohol | 1.00 |
| Dimethicone (DC 200 Fluid ® sold by the company Dow | 1.00 |
| Corning) | |
| liquid petroleum jelly | 6.00 |
| Isopropyl myristate (Estol ® IMP 1514 sold by Unichema) | 3.00 |
| Glycerol | 20.00 |
| Preserving agent | 0.30 |
| Water | qs 100 |

### Example 2D

### Facial Care Cream

| | weight % |
|---|---|
| Arachidyl behenyl alcohol/arachidylglucoside | 3.00 |
| Isohexadecane | 7.00 |
| Powder of Lactobacillus paracasei ST11 | 5.00 |
| Glycerol | 2.00 |
| Extract of Vitreoscilla filiformis | 3.00 |
| BHT | 0.05 |
| Methyl POB | 0.10 |
| Propyl POB | 0.05 |
| Water | qs 100 |

### Example 2E

### Facial Care Gel

| | weight % |
|---|---|
| Powder of Lactobacillus paracasei ST11 | 5.00 |
| Extract of Vitreoscilla filiformis | 3.00 |
| Antioxidant | 0.05 |
| Vitamin E | 2.50 |
| Isopropanol | 40.00 |
| Preserving agent | 0.30 |
| Water | qs 100 |

### Example 3

### Oral Compositions

### Example 3A

### Powder in Stick Form

| Active principle | | |
|---|---|---|
| Lactobacillus paracasei ST11 | 10¹⁰ | cfu |
| Excipient | | |
| Xanthan gum | 0.8 | mg |
| Sodium benzoate | 0.2 | mg |
| Maltodextrin | qs 30 | g |

One stick may be taken per day.

### Example 3B

### Capsule

| | mg/capsule |
|---|---|
| Lactobacillus johnsonii | 10⁸ cfu |
| Vitamin C | 60.00 |
| Magnesium stearate | 0.02 |

One to three of these wafer capsules may be taken per day.

### Example 3C

### Sugar-Coated Tablet

| | mg/tablet |
|---|---|
| Active materials | |
| Lactobacillus paracasei ST11 | 5 × 10⁸ cfu |
| Excipient for the core of the tablet | |
| Microcrystalline cellulose | 70.0 |
| Encompress ™ | 60.0 |
| Magnesium stearate | 3.0 |
| Anhydrous colloidal silica | 1.0 |
| Coating agent | |
| Shellac | 5.0 |
| Talc | 61.0 |
| Sucrose | 250.0 |
| Polyvidone | 6.0 |
| Titanium dioxide | 0.3 |
| Colorant | 5.0 |

This type of sugar-coated tablet may be taken 1 to 3 times per day.

### Example 3D

### Sugar-Coated Tablet

| | mg/tablet |
|---|---|
| Active materials | |
| Lactobacillus paracasei ST11 | 10⁹ cfu |
| Lactobacillus johnsonii | 10⁹ cfu |
| Excipient for the core of the tablet | |
| Microcrystalline cellulose | 70.0 |
| Encompress ™ | 60.0 |
| Magnesium stearate | 3.0 |
| Anhydrous colloidal silica | 1.0 |
| Coating agent | |
| Shellac | 5.0 |
| Talc | 61.0 |
| Sucrose | 250.0 |
| Polyvidone | 6.0 |
| Titanium dioxide | 0.3 |
| Colorant | 5.0 |

This type of sugar-coated tablet may be taken 1 to 3 times per day.

1-13. (canceled)

14. A cosmetic process for preventing and/or treating impaired radiance of the skin's complexion, comprising administering at least one probiotic microorganism to an individual in need thereof.

15. The process of claim 14, wherein the probiotic microorganism is of the Lactobacillus genus.

16. The process of claim 14, wherein the process is for preventing and/or treating loss of radiance of the skin's complexion.

17. The process of claim 14, wherein the process is for giving the skin a luminous complexion.

18. The process of claim 14, wherein the process is for preventing and/or treating a blurry, dull and/or non-uniform skin complexion.

19. The process of claim 14, wherein the process is for preventing and/or treating skin imperfections.

20. The process of claim 19, wherein the skin imperfections are selected from the group consisting of spots, dartres, dyschromia, age marks, blackheads and melisma.

21. The process of claim 14, wherein the probiotic microorganism is orally, topically or parenterally administered.

22. The process of claim 14, wherein the probiotic microorganism is orally administered.

23. The process of claim 14, wherein the probiotic microorganism is living, semi-activated, inactivated or dead.

24. The process of claim 14, wherein the probiotic microorganism is administered as a lysate.

25. The process of claim 14, wherein the probiotic microorganism is selected from the group consisting of Lactobacillus, Bifidobacterium, Cocci, yeasts, sporulating bacteria, and mixtures thereof.

26. The process of claim 14, wherein the probiotic microorganism is a Lactobacillus paracasei.

27. The process of claim 14, wherein the probiotic microorganism is Lactobacillus paracasei CNCM I-2116 (ST11).

28. The process of claim 14, wherein the probiotic microorganism is in a composition in a proportion of from 0.0001% to 30% by weight relative to the total weight of the composition.

29. The process of claim 14, wherein the probiotic microorganism is in a composition in a proportion of from 0.001% to 20% by weight relative to the total weight of the composition.

30. The process of claim 14, wherein the probiotic microorganism is in a composition in a proportion of from 0.01% to 15% by weight relative to the total weight of the composition.

31. The process of claim 14, wherein the probiotic microorganism is in a composition in a proportion of from 0.1% to 10% by weight relative to the total weight of the composition.

32. The process of claim 14, wherein the probiotic microorganism is in a composition in a proportion of from 1% to 5% by weight relative to the total weight of the composition.

33. The process of claim 14, wherein the probiotic microorganism is orally administered as a living probiotic microorganism in a composition in a proportion of from 10³ to 10¹⁵ cfu per gram of the composition.

34. The process of claim 14, wherein the probiotic microorganism is orally administered as a living probiotic microorganism in a composition in a proportion of from 10⁵ to 10¹⁵ cfu per gram of the composition.

35. The process of claim 14, wherein the probiotic microorganism is orally administered as a living probiotic microorganism in a composition in a proportion of from 10⁷ to 10¹² cfu per gram of the composition.

36. The process of claim 14, wherein the probiotic microorganism is administered in combination with an additional active agent.
